(19) United States
(12) Patent Application Publication
(10) Pub. No.: US 2023/0263053 A1
(43) Pub. Date: Aug. 17, 2023

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Gi-Back LEE, Yongin-si (KR); Hyun-Ju LA, Yongin-si (KR); Won-Jang JEONG, Yongin-si (KR); Dong-Jun KIM, Yongin-si (KR); Hyo-Kyun HAM, Yongin-si (KR)

(73) Assignee: LT MATERIALS CO., LTD., Yongin-si, Gyeonggi-do (KR)

(21) Appl. No.: 18/012,019

(22) PCT Filed: Mar. 10, 2021
(86) PCT No.: PCT/KR2021/002958

§ 371 Date: Dec. 21, 2022

(30) Foreign Application Priority Data
Jul. 9, 2020 (KR) 10-2020-0084746

(51) Int. Cl. H10K 85/60; C07D 213/85; C09K 11/06

(52) U.S. Cl. CPC H10K 85/654; C07D 213/85; C09K 11/06; H10K 50/17

(57) ABSTRACT

The present application provides a heterocyclic compound capable of significantly enhancing lifetime, efficiency, electrochemical stability and thermal stability of an organic light emitting device, and an organic light emitting device containing the heterocyclic compound in an organic material layer.

### TECHNICAL FIELD

This application claims priority to and the benefits of Korean Patent Application No. 10-2020-0084746, filed with the Korean Intellectual Property Office on Jul. 9, 2020, the entire contents of which are incorporated herein by reference.

The present specification relates to a heterocyclic compound, and an organic light emitting device comprising the same.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### DISCLOSURE

### Technical Problem

The present specification is directed to providing a heterocyclic compound, and an organic light emitting device comprising the same.

### Technical Solution

One embodiment of the present application provides a heterocyclic compound represented by the following Chemical Formula 1.

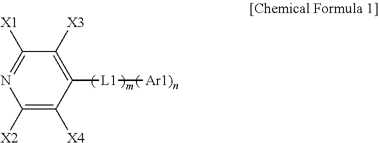

In Chemical Formula 1,

L1 is a direct bond; a substituted or unsubstituted arylene group having 6 to 60 carbon atoms; or a substituted or unsubstituted heteroarylene group having 2 to 60 carbon atoms,

two of X1 to X4 are each independently an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂, and the remaining two are each independently a halogen group; a cyano group; or −NO₂,

Ar1 is an aryl group having 6 to 60 carbon atoms unsubstituted or substituted with one or more selected from the group consisting of a halogen group, a cyano group, a haloalkyl group and −NO₂; or a heteroaryl group having 2 to 60 carbon atoms unsubstituted or substituted with one or more selected from the group consisting of a halogen group, a cyano group, a haloalkyl group and −NO₂, and

m is an integer of 0 to 2, n is an integer of 1 or 2, and when m and n are each 2, substituents in the parentheses are the same as or different from each other.

In addition, one embodiment of the present application provides an organic light emitting device comprising a first electrode; a second electrode; and one or more organic material layers provided between the first electrode and the second electrode, wherein one or more layers of the organic material layers comprise the heterocyclic compound represented by Chemical Formula 1.

### Advantageous Effects

A heterocyclic compound described in the present specification can be used as a material of an organic material layer of an organic light emitting device. The heterocyclic compound is capable of performing a role of a hole injection material, a hole transfer material, a light emitting material, an electron transfer material, an electron injection material or the like in the organic light emitting device. Particularly, the heterocyclic compound can be used as an electron transfer layer material, a hole blocking layer material or a charge generation layer material of the organic light emitting device.

Specifically, when using the heterocyclic compound represented by Chemical Formula 1 in the organic material layer, a driving voltage of the device can be lowered, light efficiency can be enhanced, and lifetime properties of the device can be enhanced.

### DESCRIPTION OF DRAWINGS

FIG. 1 to FIG. 4 are diagrams each illustrating a lamination structure of an organic light emitting device according to one embodiment of the present application.

### MODE FOR DISCLOSURE

Hereinafter, the present specification will be described in more detail.

In the present specification, a certain part “comprising” certain constituents means capable of further including other constituents, and does not exclude other constituents unless particularly stated on the contrary.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of a linear or branched alkyl group having 1 to 60 carbon atoms; a linear or branched alkenyl group having 2 to 60 carbon atoms; a linear or branched alkynyl group having 2 to 60 carbon atoms; a monocyclic or polycyclic cycloalkyl group having 3 to 60 carbon atoms; a monocyclic or polycyclic heterocycloalkyl group having 2 to 60 carbon atoms; a monocyclic or polycyclic aryl group having 6 to 60 carbon atoms; a monocyclic or polycyclic heteroaryl group having 2 to 60 carbon atoms; a silyl group; a phosphine oxide group; and an amine group, or being unsubstituted, or being substituted with a substituent linking two or more substituents selected from among the substituents illustrated above, or being unsubstituted.

More specifically, “substituted or unsubstituted” in the present specification means being substituted with one or more substituents selected from the group consisting of a monocyclic or polycyclic aryl group having 6 to 60 carbon atoms; or a monocyclic or polycyclic heteroaryl group having 2 to 60 carbon atoms.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the haloalkyl group represents an alkyl group as defined in the present disclosure having one, two or more hydrogen atoms replaced by the same or different halogen atoms. The term “haloalkyl” also comprises a perhalogenated alkyl group having all alkyl hydrogen atoms replaced by halogen atoms. The preferred haloalkyl group may comprise, but is not limited to, —CH₂Cl, —CF₃, —CH₂CF₃, —CH₂CCl₃ and the like.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the fluorenyl group may be substituted, and adjacent substituents may bond to each other to form a ring.

In the present specification, the Spiro group is a group comprising a spiro structure, and may have 15 to 60 carbon atoms. For example, the spiro group may comprise a structure in which a 2,3-dihydro-1H-indene group or a cyclohexane group spiro bonds to a fluorenyl group. Specifically, the following spiro group may comprise any one of groups of the following structural formulae.

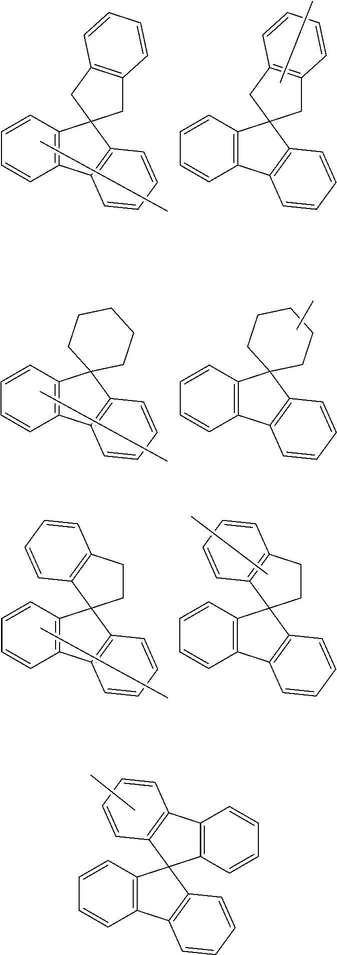

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25.

Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the arylene group means the aryl group having two bonding sites, that is, a divalent group.

The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

One embodiment of the present application provides a heterocyclic compound represented by the following Chemical Formula 1.

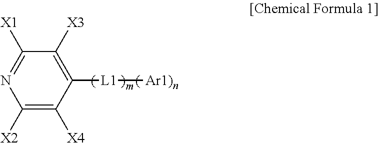

In Chemical Formula 1,

L1 is a direct bond; a substituted or unsubstituted arylene group having 6 to 60 carbon atoms; or a substituted or unsubstituted heteroarylene group having 2 to 60 carbon atoms,

two of X1 to X4 are each independently an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂, and the remaining two are each independently a halogen group; a cyano group; or −NO₂,

Ar1 is an aryl group having 6 to 60 carbon atoms unsubstituted or substituted with one or more selected from the group consisting of a halogen group, a cyano group, a haloalkyl group and −NO₂; or a heteroaryl group having 2 to 60 carbon atoms unsubstituted or substituted with one or more selected from the group consisting of a halogen group, a cyano group, a haloalkyl group and −NO₂, and

m is an integer of 0 to 2, n is an integer of 1 or 2, and when m and n are each 2, substituents in the parentheses are the same as or different from each other.

The heterocyclic compound represented by Chemical Formula 1 has increased electron withdrawing properties by bonding a specific substituent to the pyridine structure, and has excellent efficiency and driving through adjusting a band gap and a T1 value. Excitons in a light emitting area increases when forming proper energy level and band gap, and having increased excitons in a light emitting area means having effects of increasing driving voltage and efficiency of a device. In addition, a long lifetime device with superior hole transfer ability and thermal stability is obtained by having a high T1 value. The T1 value herein means an energy level value in a triplet state.

Particularly, excellent electron withdrawing properties of a functional group such as pyridine and a cyano group attract electrons to break bonds between holes and electron pairs, and as a result, a property of facilitating hole generation is obtained and a property of enhancing hole mobility is also obtained.

In one embodiment of the present application, L1 may be a direct bond; a substituted or unsubstituted arylene group having 6 to 60 carbon atoms; or a substituted or unsubstituted heteroarylene group having 2 to 60 carbon atoms.

In another embodiment, L1 may be a direct bond; a substituted or unsubstituted arylene group having 6 to 40 carbon atoms; or a substituted or unsubstituted heteroarylene group having 2 to 40 carbon atoms.

In another embodiment, L1 may be a direct bond; a substituted or unsubstituted arylene group having 6 to 20 carbon atoms; or a substituted or unsubstituted heteroarylene group having 2 to 20 carbon atoms.

In another embodiment, L1 may be a direct bond; a substituted or unsubstituted phenylene group; a substituted or unsubstituted biphenylene group; a substituted or unsubstituted naphthylene group; or a substituted or unsubstituted anthracenyl group.

In another embodiment, L1 may be a direct bond; a phenylene group; a biphenylene group; a naphthylene group; or an anthracenyl group.

In another embodiment, L1 is a direct bond.

In one embodiment of the present application, X1 to X4 of Chemical Formula 1 are the same as or different from each other, and may be each independently a halogen group; a cyano group; a haloalkyl group; −NO₂; an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂.

In one embodiment of the present application, X1 to X4 are the same as or different from each other, and may be each independently a halogen group; a cyano group; a haloalkyl group; −NO₂; an aryl group having 6 to 20 carbon atoms unsubstituted or substituted with one or more selected from the group consisting of a halogen group, a cyano group, a haloalkyl group and −NO₂; or a heteroaryl group having 2 to 20 carbon atoms unsubstituted or substituted with one or more selected from the group consisting of a halogen group, a cyano group, a haloalkyl group and −NO₂.

In one embodiment of the present application, X1 to X4 are the same as or different from each other, and may be each independently a halogen group; a cyano group; a haloalkyl group; −NO₂; an aryl group having 6 to 20 carbon atoms substituted with one or more selected from the group consisting of a halogen group, a cyano group, a haloalkyl group and −NO₂; or a heteroaryl group having 2 to 20 carbon atoms unsubstituted or substituted with one or more selected from the group consisting of a halogen group, a cyano group, a haloalkyl group and −NO₂.

In one embodiment of the present application, X1 to X4 are the same as or different from each other, and may be each independently a halogen group; a cyano group; a haloalkyl group; −NO₂; a phenyl group substituted with one or more selected from the group consisting of a halogen group, a cyano group, a haloalkyl group and −NO₂; or a pyridine group unsubstituted or substituted with one or more selected from the group consisting of a halogen group, a cyano group, a haloalkyl group and −NO₂.

In one embodiment of the present application, X1 to X4 are the same as or different from each other, and may be each independently a halogen group; a cyano group; a haloalkyl group; −NO₂; a phenyl group substituted with one or more selected from the group consisting of a halogen group, a cyano group, −CF₃ and −NO₂; or a pyridine group unsubstituted or substituted with one or more selected from the group consisting of a halogen group, a cyano group, —CF₃ and −NO₂.

In one embodiment of the present application, two of X1 to X4 may be each independently an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂, and the remaining two may be each independently a halogen group; a cyano group; a haloalkyl group; or −NO₂.

In one embodiment of the present application, two of X1 to X4 may be each independently an aryl group having 6 to 20 carbon atoms unsubstituted or substituted with one or more selected from the group consisting of a halogen group, a cyano group, a haloalkyl group and −NO₂; or a heteroaryl group having 2 to 20 carbon atoms unsubstituted or substituted with one or more selected from the group consisting of a halogen group, a cyano group, a haloalkyl group and −NO₂, and the remaining two may be each independently a halogen group; a cyano group; a haloalkyl group; or −NO₂.

In one embodiment of the present application, two of X1 to X4 may be each independently a phenyl group substituted with one or more selected from the group consisting of a halogen group, a cyano group, a haloalkyl group and −NO₂; or a pyridine group having 2 to 20 carbon atoms unsubstituted or substituted with one or more selected from the group consisting of a halogen group, a cyano group, a haloalkyl group and −NO₂, and the remaining two may be each independently a halogen group; a cyano group; a haloalkyl group; or −NO₂.

In one embodiment of the present application, two of X1 to X4 may be each independently a phenyl group substituted with one or more selected from the group consisting of a halogen group, a cyano group, —CF₃ and −NO₂; or a pyridine group having 2 to 20 carbon atoms unsubstituted or substituted with one or more selected from the group consisting of a halogen group, a cyano group, —CF₃ and −NO₂, and the remaining two may be each independently a halogen group; a cyano group; —CF₃; or −NO₂.

In one embodiment of the present application, X1 and X2 may be the same as each other, and X3 and X4 may be the same as each other.

In one embodiment of the present application, X1 and X2 may be the same as each other, X3 and X4 may be the same as each other, and X1 and X3 may be different from each other.

In one embodiment of the present application, Ar1 of Chemical Formula 1 may be an aryl group having 6 to 60 carbon atoms unsubstituted or substituted with one or more selected from the group consisting of a halogen group, a cyano group, a haloalkyl group and −NO₂; or a heteroaryl group having 2 to 60 carbon atoms unsubstituted or substituted with one or more selected from the group consisting of a halogen group, a cyano group, a haloalkyl group and −NO₂.

In one embodiment of the present application, Ar1 of Chemical Formula 1 may be an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂.

In one embodiment of the present application, Ar1 of Chemical Formula 1 may be an aryl group having 6 to 20 carbon atoms unsubstituted or substituted with one or more selected from the group consisting of a halogen group, a cyano group, a haloalkyl group and −NO₂; or a heteroaryl group having 2 to 20 carbon atoms unsubstituted or substituted with one or more selected from the group consisting of a halogen group, a cyano group, a haloalkyl group and −NO₂.

In one embodiment of the present application, Ar1 of Chemical Formula 1 may be an aryl group having 6 to 20 carbon atoms substituted with one or more selected from the group consisting of a halogen group, a cyano group, a haloalkyl group and −NO₂; or a heteroaryl group having 2 to 20 carbon atoms unsubstituted or substituted with one or more selected from the group consisting of a halogen group, a cyano group, a haloalkyl group and −NO₂.

In one embodiment of the present application, Ar1 of Chemical Formula 1 may be a phenyl group substituted with one or more selected from the group consisting of a halogen group, a cyano group, a haloalkyl group and −NO₂; or a pyridine group unsubstituted or substituted with one or more selected from the group consisting of a halogen group, a cyano group, a haloalkyl group and −NO₂.

In one embodiment of the present application, Ar1 of Chemical Formula 1 may be a phenyl group substituted with one or more selected from the group consisting of a halogen group, a cyano group, —CF₃ and −NO₂; or a pyridine group unsubstituted or substituted with one or more selected from the group consisting of a halogen group, a cyano group, —CF₃ and −NO₂.

In one embodiment of the present application, Chemical Formula 1 may be represented by the following Chemical Formula 2 or 3.

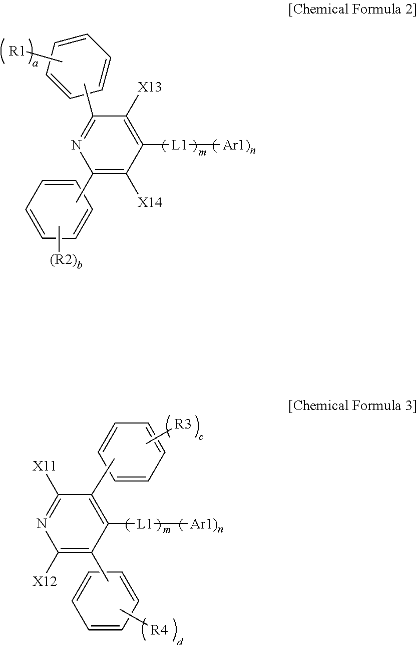

In Chemical Formulae 2 and 3,

L1, Ar1, m and n have the same definitions as in Chemical Formula 1,

X11 to X14 and R1 to R4 are the same as or different from each other, and each independently a halogen group; a cyano group; a haloalkyl group; or −NO₂, and

a to d are each independently an integer of 1 to 5, and when a to d are each 2 or greater, substituents in the parentheses are the same as or different from each other.

In one embodiment of the present application, Chemical Formula 1 may be represented by the following Chemical Formula 4 or 5.

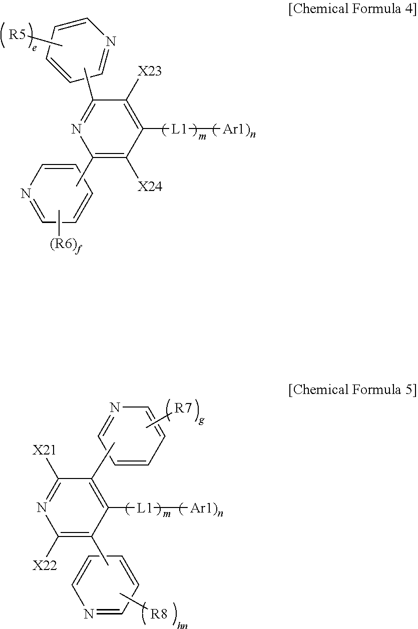

In Chemical Formulae 4 and 5,

L1, Ar1, m and n have the same definitions as in Chemical Formula 1,

X21 to X24 are the same as or different from each other, and each independently a halogen group; a cyano group; a haloalkyl group; or −NO₂,

R5 to R8 are the same as or different from each other, and each independently hydrogen; deuterium; a halogen group; a cyano group; a haloalkyl group; or −NO₂, and

e to h are each independently an integer of 1 to 4, and when e to h are each 2 or greater, substituents in the parentheses are the same as or different from each other.

In one embodiment of the present application, X11 and X12 are the same as each other.

In one embodiment of the present application, X13 and X14 are the same as each other.

In one embodiment of the present application, X21 and X22 are the same as each other.

In one embodiment of the present application, X23 and X24 are the same as each other.

In one embodiment of the present application, X11 to X14 are the same as or different from each other, and may be each independently a halogen group; a cyano group; a haloalkyl group; or −NO₂.

In one embodiment of the present application, X11 to X14 are the same as or different from each other, and may be each independently a halogen group; a cyano group; —CF₃; or −NO₂.

*92 In one embodiment of the present application, X21 to X24 are the same as or different from each other, and may be each independently a halogen group; a cyano group; a haloalkyl group; or −NO₂.

In one embodiment of the present application, X21 to X24 are the same as or different from each other, and may be each independently a halogen group; a cyano group; —CF₃; or −NO₂.

In one embodiment of the present application, R1 to R4 are the same as or different from each other, and may be each independently a halogen group; a cyano group; a haloalkyl group; or −NO₂.

In one embodiment of the present application, R1 to R4 are the same as or different from each other, and may be each independently a halogen group; a cyano group; —CF₃; or −NO₂.

In one embodiment of the present application, R5 to R8 are the same as or different from each other, and may be each independently hydrogen; deuterium; a halogen group; a cyano group; a haloalkyl group; or −NO₂.

In one embodiment of the present application, R5 to R8 are the same as or different from each other, and may be each independently hydrogen; deuterium; a halogen group; a cyano group; —CF₃; or −NO₂.

In the heterocyclic compound provided in one embodiment of the present application, Chemical Formula 1 is represented by any one of the following compounds.

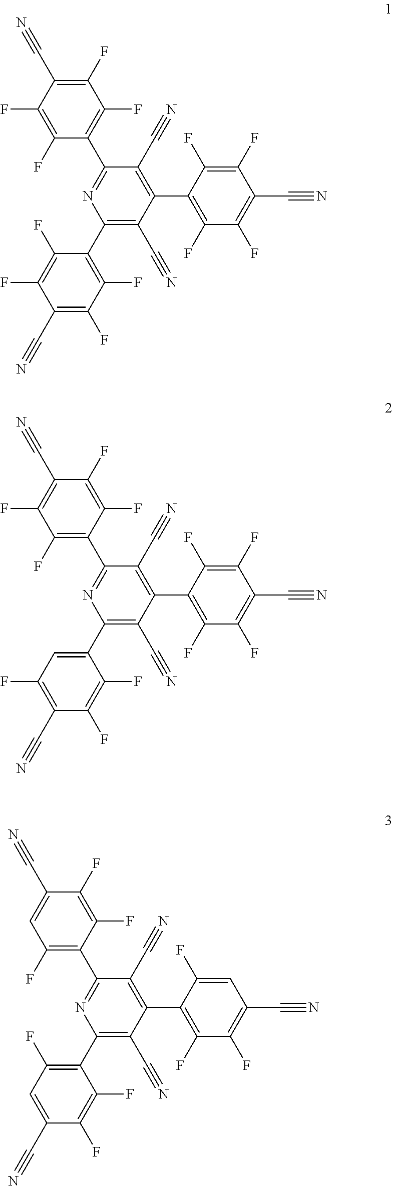 [SMILES: N#Cc1c(F)c(F)c(-c2nc(-c3c(F)c(F)c(C#N)c(F)c3F)c(C#N)c(-c3c(F)c(F)c(C#N)c(F)c3F)c2C#N)c(F)c1F ; N#Cc1c(F)cc(-c2nc(-c3c(F)c(F)c(C#N)c(F)c3F)c(C#N)c(-c3c(F)c(F)c(C#N)c(F)c3F)c2C#N)c(F)c1F ; N#Cc1cc(F)c(-c2nc(-c3c(F)cc(C#N)c(F)c3F)c(C#N)c(-c3c(F)cc(C#N)c(F)c3F)c2C#N)c(F)c1F]

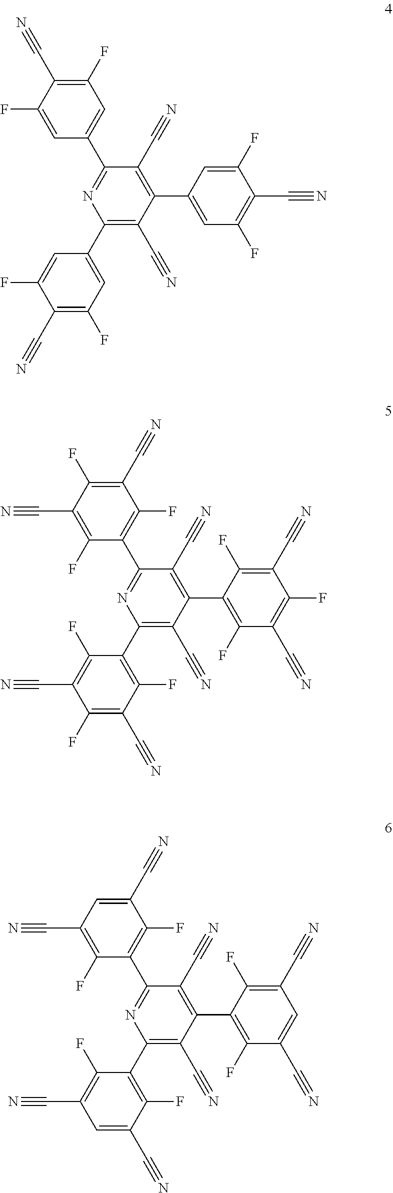 [SMILES: N#Cc1c(F)cc(-c2nc(-c3cc(F)c(C#N)c(F)c3)c(C#N)c(-c3cc(F)c(C#N)c(F)c3)c2C#N)cc1F ; N#Cc1c(F)c(C#N)c(F)c(-c2nc(-c3c(F)c(C#N)c(F)c(C#N)c3F)c(C#N)c(-c3c(F)c(C#N)c(F)c(C#N)c3F)c2C#N)c1F ; N#Cc1cc(C#N)c(F)c(-c2nc(-c3c(F)c(C#N)cc(C#N)c3F)c(C#N)c(-c3c(F)c(C#N)cc(C#N)c3F)c2C#N)c1F]

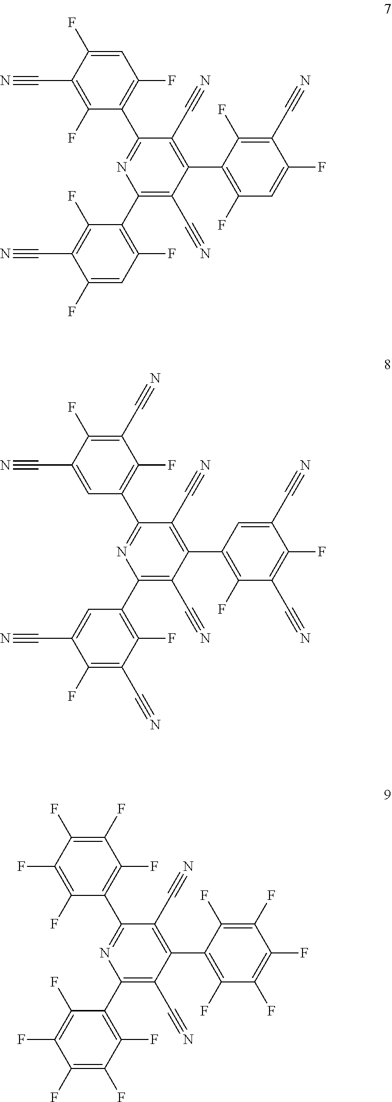 [SMILES: N#Cc1c(F)cc(F)c(-c2nc(-c3c(F)cc(F)c(C#N)c3F)c(C#N)c(-c3c(F)cc(F)c(C#N)c3F)c2C#N)c1F ; N#Cc1cc(-c2nc(-c3cc(C#N)c(F)c(C#N)c3F)c(C#N)c(-c3cc(C#N)c(F)c(C#N)c3F)c2C#N)c(F)c(C#N)c1F ; N#Cc1c(-c2c(F)c(F)c(F)c(F)c2F)nc(-c2c(F)c(F)c(F)c(F)c2F)c(C#N)c1-c1c(F)c(F)c(F)c(F)c1F]

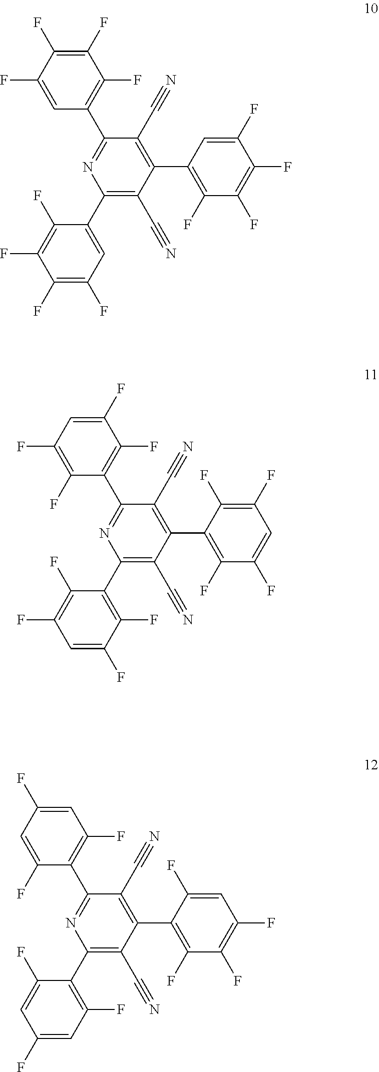 [SMILES: N#Cc1c(-c2cc(F)c(F)c(F)c2F)nc(-c2cc(F)c(F)c(F)c2F)c(C#N)c1-c1cc(F)c(F)c(F)c1F ; N#Cc1c(-c2c(F)c(F)cc(F)c2F)nc(-c2c(F)c(F)cc(F)c2F)c(C#N)c1-c1c(F)c(F)cc(F)c1F ; N#Cc1c(-c2c(F)cc(F)cc2F)nc(-c2c(F)cc(F)cc2F)c(C#N)c1-c1c(F)cc(F)c(F)c1F]

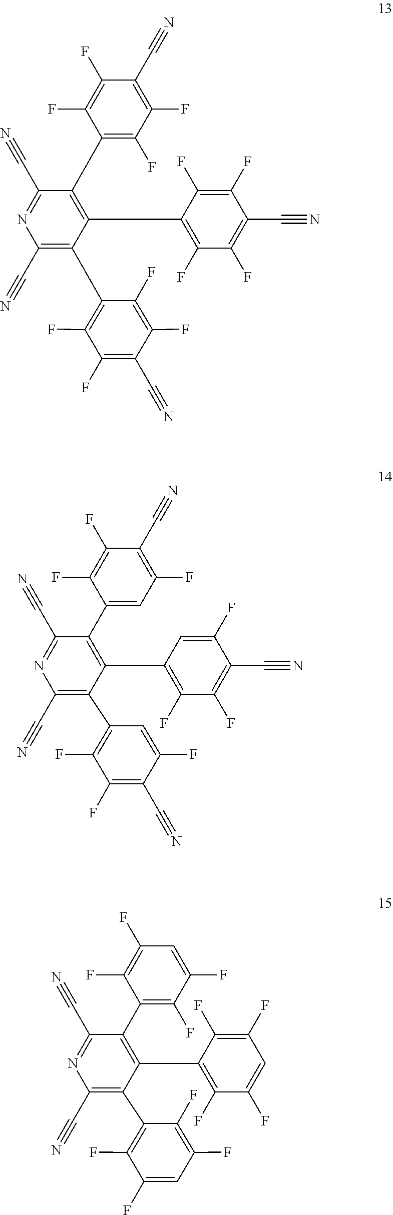 [SMILES: N#Cc1nc(C#N)c(-c2c(F)c(F)c(C#N)c(F)c2F)c(-c2c(F)c(F)c(C#N)c(F)c2F)c1-c1c(F)c(F)c(C#N)c(F)c1F ; N#Cc1nc(C#N)c(-c2cc(F)c(C#N)c(F)c2F)c(-c2cc(F)c(C#N)c(F)c2F)c1-c1cc(F)c(C#N)c(F)c1F ; N#Cc1nc(C#N)c(-c2c(F)c(F)cc(F)c2F)c(-c2c(F)c(F)cc(F)c2F)c1-c1c(F)c(F)cc(F)c1F]

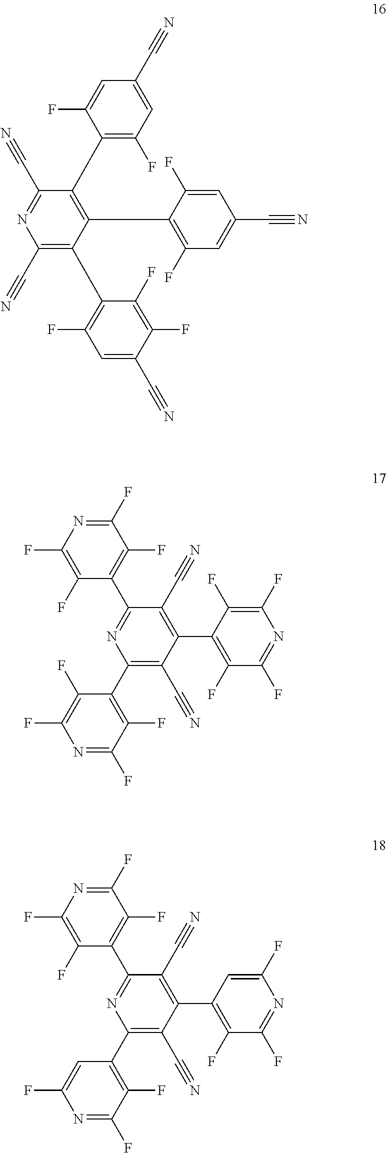 [SMILES: N#Cc1cc(F)c(-c2c(C#N)nc(C#N)c(-c3c(F)cc(C#N)c(F)c3F)c2-c2c(F)cc(C#N)cc2F)c(F)c1 ; N#Cc1c(-c2c(F)c(F)nc(F)c2F)nc(-c2c(F)c(F)nc(F)c2F)c(C#N)c1-c1c(F)c(F)nc(F)c1F ; N#Cc1c(-c2cc(F)nc(F)c2F)nc(-c2c(F)c(F)nc(F)c2F)c(C#N)c1-c1cc(F)nc(F)c1F]

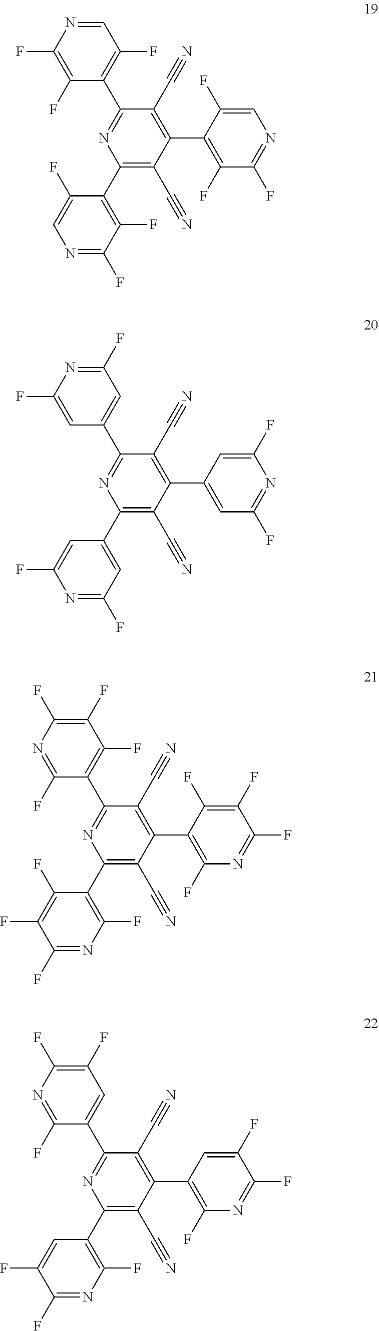 [SMILES: N#Cc1c(-c2c(F)cnc(F)c2F)nc(-c2c(F)cnc(F)c2F)c(C#N)c1-c1c(F)cnc(F)c1F ; N#Cc1c(-c2cc(F)nc(F)c2)nc(-c2cc(F)nc(F)c2)c(C#N)c1-c1cc(F)nc(F)c1 ; N#Cc1c(-c2c(F)nc(F)c(F)c2F)nc(-c2c(F)nc(F)c(F)c2F)c(C#N)c1-c1c(F)nc(F)c(F)c1F ; N#Cc1c(-c2cc(F)c(F)nc2F)nc(-c2cc(F)c(F)nc2F)c(C#N)c1-c1cc(F)c(F)nc1F]

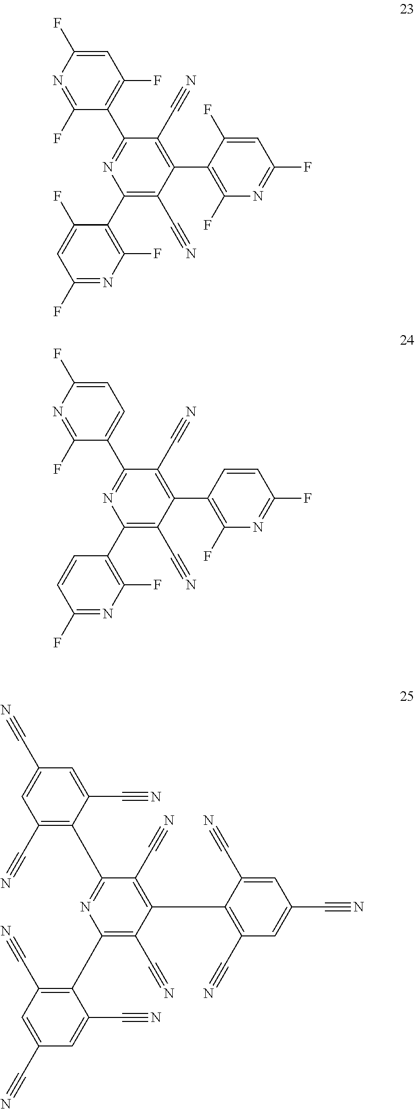

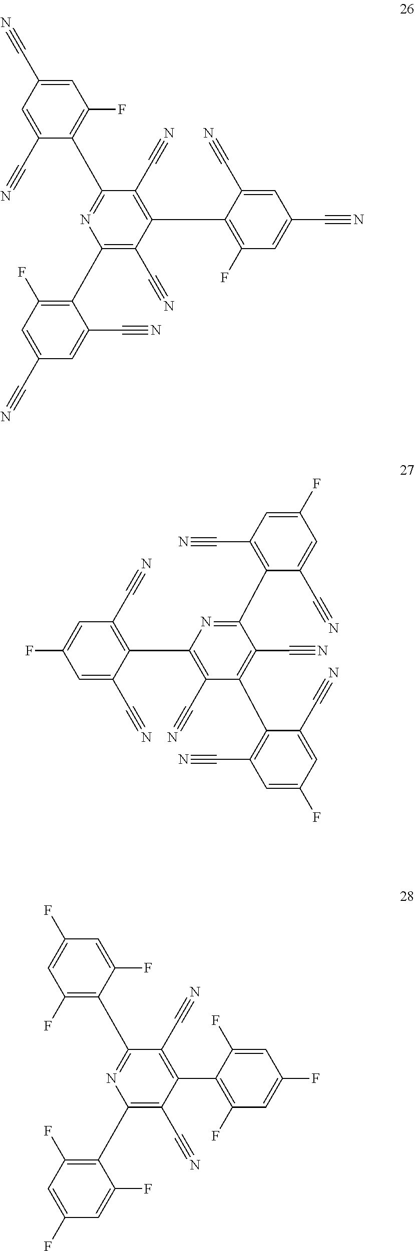 [SMILES: N#Cc1cc(F)c(-c2nc(-c3c(F)cc(C#N)cc3C#N)c(C#N)c(-c3c(F)cc(C#N)cc3C#N)c2C#N)c(C#N)c1 ; N#Cc1cc(F)cc(C#N)c1-c1nc(-c2c(C#N)cc(F)cc2C#N)c(C#N)c(-c2c(C#N)cc(F)cc2C#N)c1C#N ; N#Cc1c(-c2c(F)cc(F)cc2F)nc(-c2c(F)cc(F)cc2F)c(C#N)c1-c1c(F)cc(F)cc1F]

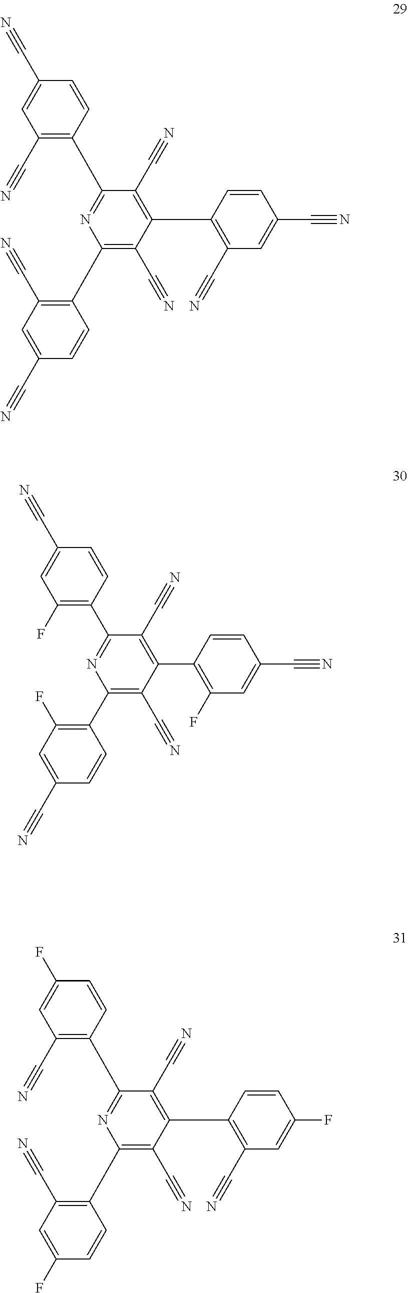 [SMILES: N#Cc1ccc(-c2nc(-c3ccc(C#N)cc3C#N)c(C#N)c(-c3ccc(C#N)cc3C#N)c2C#N)c(C#N)c1 ; N#Cc1ccc(-c2nc(-c3ccc(C#N)cc3F)c(C#N)c(-c3ccc(C#N)cc3F)c2C#N)c(F)c1 ; N#Cc1cc(F)ccc1-c1nc(-c2ccc(F)cc2C#N)c(C#N)c(-c2ccc(F)cc2C#N)c1C#N]

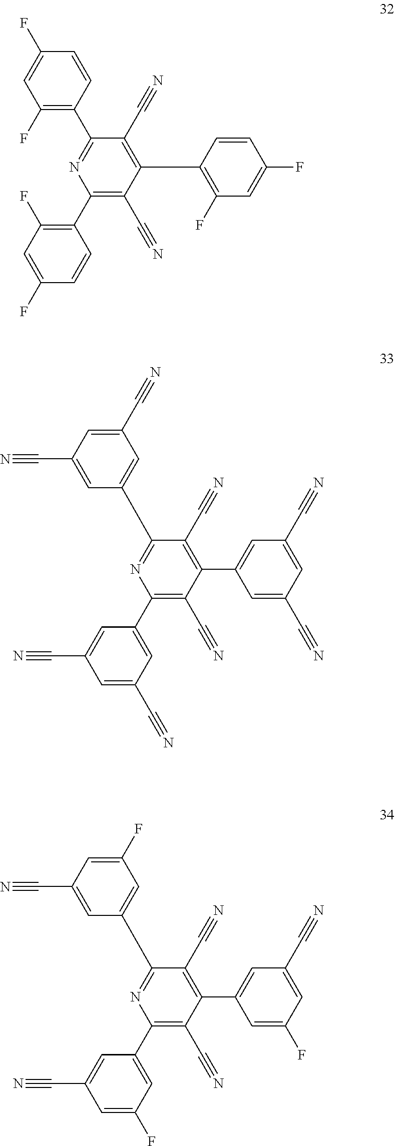 [SMILES: N#Cc1c(-c2ccc(F)cc2F)nc(-c2ccc(F)cc2F)c(C#N)c1-c1ccc(F)cc1F ; N#Cc1cc(C#N)cc(-c2nc(-c3cc(C#N)cc(C#N)c3)c(C#N)c(-c3cc(C#N)cc(C#N)c3)c2C#N)c1 ; N#Cc1cc(F)cc(-c2nc(-c3cc(F)cc(C#N)c3)c(C#N)c(-c3cc(F)cc(C#N)c3)c2C#N)c1]

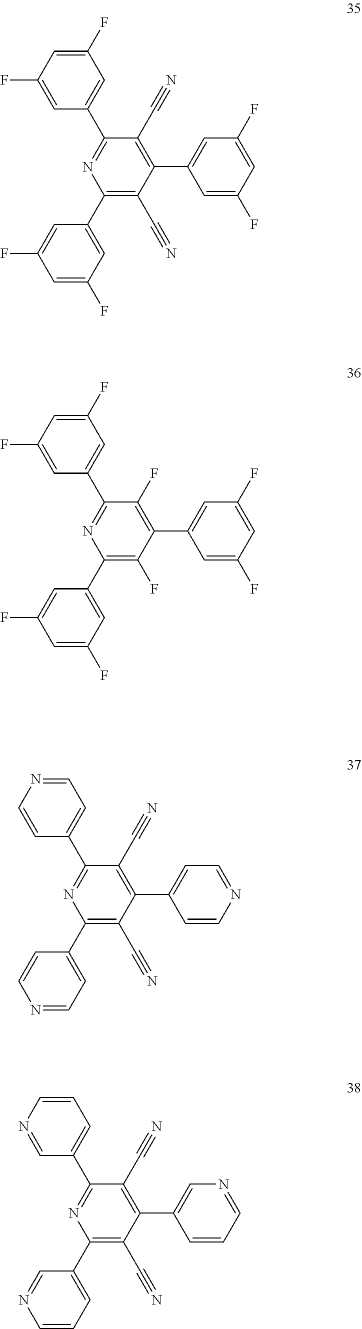 [SMILES: N#Cc1c(-c2cc(F)cc(F)c2)nc(-c2cc(F)cc(F)c2)c(C#N)c1-c1cc(F)cc(F)c1 ; Fc1cc(F)cc(-c2nc(-c3cc(F)cc(F)c3)c(F)c(-c3cc(F)cc(F)c3)c2F)c1 ; N#Cc1c(-c2ccncc2)nc(-c2ccncc2)c(C#N)c1-c1ccncc1 ; N#Cc1c(-c2cccnc2)nc(-c2cccnc2)c(C#N)c1-c1cccnc1]

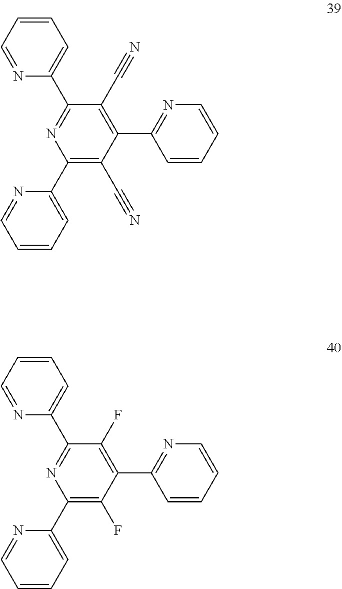 [SMILES: N#Cc1c(-c2ccccn2)nc(-c2ccccn2)c(C#N)c1-c1ccccn1 ; Fc1c(-c2ccccn2)nc(-c2ccccn2)c(F)c1-c1ccccn1]

In addition, by introducing various substituents to the structure of Chemical Formula 1, heterocyclic compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the heterocyclic compound has a high glass transition temperature (Tg) and thereby has superior thermal stability. Such an increase in the thermal stability becomes an important factor in providing driving stability to a device.

The heterocyclic compound according to one embodiment of the present application may be prepared using a multi-step chemical reaction. Some intermediate compounds are prepared first, and from the intermediate compounds, the heterocyclic compound of Chemical Formula 1 may be prepared. More specifically, the heterocyclic compound according to one embodiment of the present application may be prepared based on preparation examples to describe later.

Another embodiment of the present application provides an organic light emitting device comprising the heterocyclic compound represented by Chemical Formula 1. The “organic light emitting device” may be expressed in terms such as an “organic light emitting diode”, an “OLED”, an “OLED device” and an “organic electroluminescent device”.

One embodiment of the present application provides an organic light emitting device comprising a first electrode; a second electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment of the present application, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In another embodiment of the present application, the organic light emitting device may be a green organic light emitting device, and the heterocyclic compound according to Chemical Formula 1 may be used as a material of the green organic light emitting device.

In another embodiment of the present application, the organic light emitting device may be a red organic light emitting device, and the heterocyclic compound according to Chemical Formula 1 may be used as a material of the red organic light emitting device.

Specific descriptions on the heterocyclic compound represented by Chemical Formula 1 are the same as the descriptions provided above.

The organic light emitting device of the present application may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present application may be formed in a single layer structure, but may be formed in a multilayer structure in which two or more organic material layers are laminated. For example, the organic light emitting device of the present disclosure may have a structure comprising a hole injection layer, a hole transfer layer, a hole auxiliary layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present application, the organic material layer comprises a charge generation layer and a hole injection layer, and the charge generation layer and the hole injection layer may comprise the heterocyclic compound. When using the heterocyclic compound in the charge generation layer and the hole injection layer, proper energy level and band gap are formed increasing excitons in a light emitting area, and driving voltage and efficiency are enhanced in the device. In addition, a long lifetime device with excellent hole transfer ability and thermal stability may be obtained by having a high T1 value.

The organic light emitting device of the present disclosure may further comprise one, two or more layers selected from the group consisting of a charge generation layer, a light emitting layer, a hole injection layer, a hole transfer layer, an electron injection layer, an electron transfer layer, a hole auxiliary layer and a hole blocking layer.

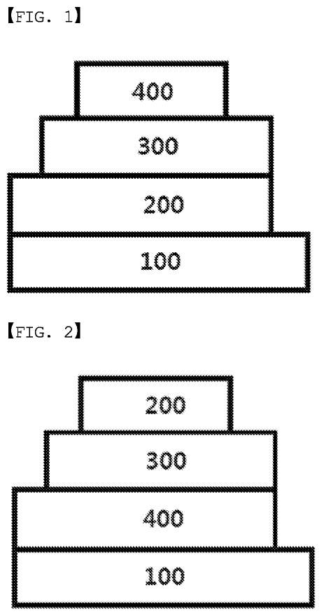

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

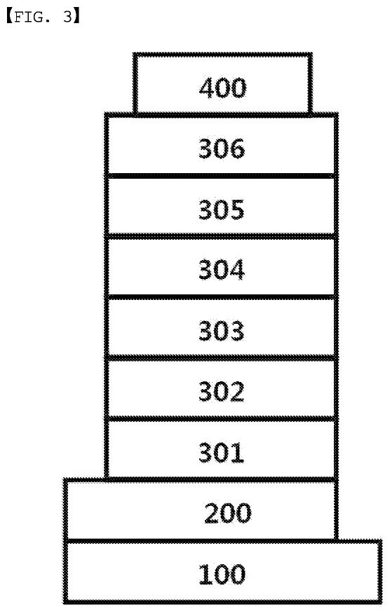

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

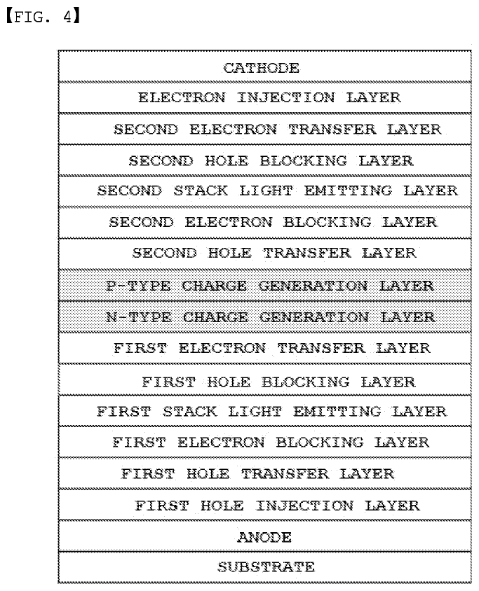

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer comprising the heterocyclic compound represented by Chemical Formula 1 may further comprise other materials as necessary.

The organic material layer according to one embodiment of the present application may comprise a first stack provided on the first electrode and comprising a first light emitting layer; a charge generation layer provided on the first stack; and a second stack provided on the charge generation layer and comprising a second light emitting layer.

In addition, the organic light emitting device according to one embodiment of the present application comprises a first electrode; a first stack provided on the first electrode and comprising a first light emitting layer; a charge generation layer provided on the first stack; a second stack provided on the charge generation layer and comprising a second light emitting layer; and a second electrode provided on the second stack.

Herein, the charge generation layer may comprise the heterocyclic compound represented by Chemical Formula 1. When using the heterocyclic compound in the charge generation layer, the organic light emitting device may have superior driving, efficiency and lifetime.

In one embodiment of the present application, the charge generation layer may be a P-type charge generation layer.

In addition, the first stack and the second stack may each independently further comprise one or more types of the hole injection layer, the hole transfer layer, the hole blocking layer, the electron transfer layer, the electron injection layer and the like described above.

As the organic light emitting device according to one embodiment of the present application, an organic light emitting device having a 2-stack tandem structure is illustrated in FIG. 4.

Herein, the first electron blocking layer, the first hole blocking layer, the second hole blocking layer and the like described in FIG. 4 may not be included in some cases.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

In the organic light emitting device of the present application, the organic material layer comprises a light emitting layer, and the light emitting layer may comprise the heterocyclic compound as a host material of a light emitting material.

In the organic light emitting device of the present application, the light emitting layer may comprise two or more host materials, and at least one of the host materials may comprise the heterocyclic compound as a host material of a light emitting material.

In the organic light emitting device of the present application, the light emitting layer may comprise two or more host materials, the two or more host materials each comprise one or more p-type host materials and n-type host materials, and at least one of the host materials may comprise the heterocyclic compound as a host material of a light emitting material. In this case, the organic light emitting device may have superior driving, efficiency and lifetime.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE 1

### Preparation of Compound 1

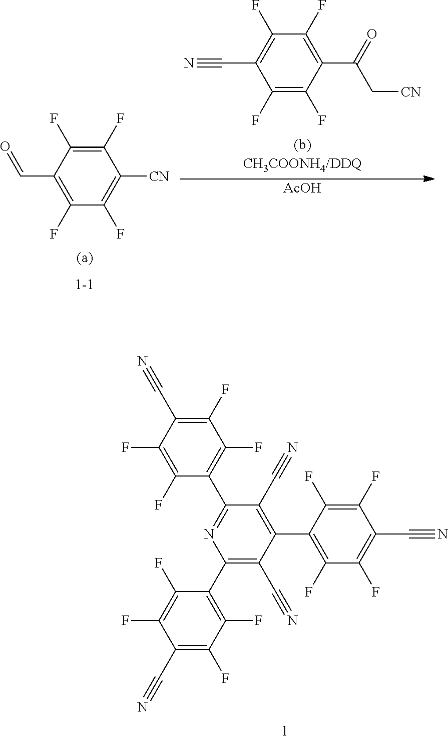 [SMILES: "AcOH" ; N#Cc1c(F)c(F)c(C=O)c(F)c1F ; N#CCC(=O)c1c(F)c(F)c(C#N)c(F)c1F ; N#Cc1c(F)c(F)c(-c2nc(-c3c(F)c(F)c(C#N)c(F)c3F)c(C#N)c(-c3c(F)c(F)c(C#N)c(F)c3F)c2C#N)c(F)c1F]

### 1) Preparation of Compound 1

Intermediate (a) (1 g, 0.0049 mol, 1 eq.), Intermediate (b) (3 g, 0.012 mol, 2.5 eq.), ammonium acetate (3.8 g, 0.049 mol, 10 eq.) and glacial acetic acid (10 ml) were introduced, and stirred for 5 hours under reflux. Solids produced after lowering the temperature were filtered, and after introducing glacial acetic acid (10 ml) and 2,3-dichloro-5,6-dicyano-1,4-benzoquinone (DDQ) (2.8 g, 0.012 mol, 2.5 eq.) thereto, the result was stirred for 1 hour (h) under reflux. Produced solids were filtered to obtain Compound 1 (1.2 g) in a 37% yield.

Target compounds were synthesized in the same manner as in Preparation Example 1 using Intermediates A and B of the following Table 1 instead of (a) and (b).

TABLE 1

| Target | | | |
|---|---|---|---|
| Compound | Intermediate A | Intermediate B | Yield |
| 2 | 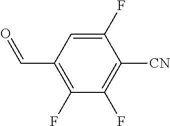 [SMILES: N#Cc1c(F)cc(C=O)c(F)c1F] | 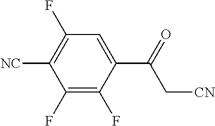 [SMILES: N#CCC(=O)c1cc(F)c(C#N)c(F)c1F] | 32% |
| 4 | 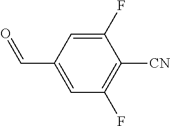 [SMILES: N#Cc1c(F)cc(C=O)cc1F] | 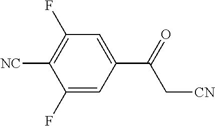 [SMILES: N#CCC(=O)c1cc(F)c(C#N)c(F)c1] | 30% |
| 6 | 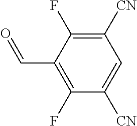 [SMILES: N#Cc1cc(C#N)c(F)c(C=O)c1F] | 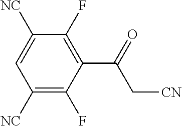 [SMILES: N#CCC(=O)c1c(F)c(C#N)cc(C#N)c1F] | 26% |
| 9 | 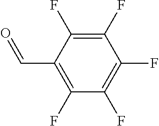 [SMILES: O=Cc1c(F)c(F)c(F)c(F)c1F] | 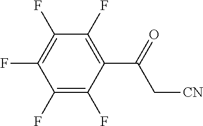 [SMILES: N#CCC(=O)c1c(F)c(F)c(F)c(F)c1F] | 28% |
| 11 | 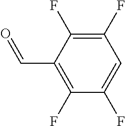 [SMILES: O=Cc1c(F)c(F)cc(F)c1F] | 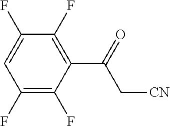 [SMILES: N#CCC(=O)c1c(F)c(F)cc(F)c1F] | 36% |
| 17 | 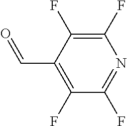 [SMILES: O=Cc1c(F)c(F)nc(F)c1F] | 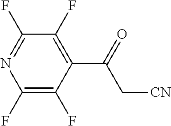 [SMILES: N#CCC(=O)c1c(F)c(F)nc(F)c1F] | 33% |
| 20 | 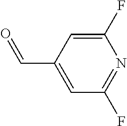 | 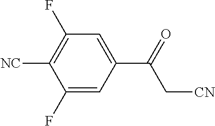 [SMILES: N#CCC(=O)c1cc(F)c(C#N)c(F)c1] | 30% |
| 33 | 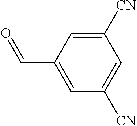 [SMILES: N#Cc1cc(C#N)cc(C=O)c1] | 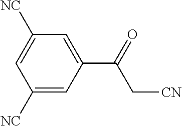 [SMILES: N#CCC(=O)c1cc(C#N)cc(C#N)c1] | 30% |
| 38 | 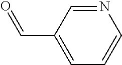 | 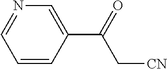 [SMILES: N#CCC(=O)c1cccnc1] | 29% |

Compounds were prepared in the same manner as in the preparation examples, and the synthesis identification results are shown in the following Table 2 and Table 3. The following Table 2 shows measurement values of ¹H NMR (CDCl₃, 200 Mz), and the following Table 3 shows measurement values of FD-mass spectrometry (FD-MS: field desorption mass spectrometry).

TABLE 2

| Compound | ¹H NMR (CDCl₃, 200 Mz) |
|---|---|
| 1 | — |
| 2 | δ = 7.96 (m, 2H), 7.45 (m, 1H) |
| 4 | δ = 7.98 (m, 4H), 7.47 (d, 2H) |
| 6 | δ = 7.49 (m, 3H) |
| 9 | — |
| 11 | δ = 6.66 (m, 3H) |
| 17 | — |
| 20 | δ = 8.13 (m, 4H), 7.62 (d, 2H) |
| 33 | δ = 8.52 (s, 4H), 8.01 (s, 2H), 7.53 |
| | (s, 2H), 7.47 (s, 1H) |
| 38 | δ = 9.75 (s, 2H) , 9.24 (s, 1H), 8.93 |
| | (m, 2H), 8.70 (m, 3H), 8.42 (m, 1H), |
| | 7.57 (m, 3H) |

TABLE 3

| Compound | FD-MS |
|---|---|
| 1 | m/z = 648.00 |
| | (C28F12N6 = 648.32) |
| 2 | m/z = 594.03 |
| | (C28H3F9N6 = 594.35) |
| 4 | m/z = 540.06 |
| | (C28H6F6N6 = 540.38) |
| 6 | m/z = 615.04 |
| | (C31H3F6N9 = 615.41) |
| 9 | m/z = 626.99 |
| | (C25F15N3 = 627.26) |
| 11 | m/z = 573.01 |
| | (C25H3F12N3 = 573.29) |
| 17 | m/z = 576.00 |
| | (C22F12N6 = 576.26) |
| 20 | m/z = 468.06 |
| | (C22H6F6N6 = 468.31) |
| 33 | m/z = 507.10 |
| | (C31H9N9 = 507.46) |
| 38 | m/z = 360.11 |
| | (C22H12N6 = 360.37) |

### EXPERIMENTAL EXAMPLE

### Experimental Example 1

1) Manufacture of Organic Light Emitting Device

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition. On the transparent ITO electrode (anode), organic materials were formed in a 2-stack white organic light emitting device (WOLED) structure.

As for the first stack, TAPC was thermal vacuum deposited first to a thickness of 300 Å to form a hole transfer layer. After forming the hole transfer layer, a light emitting layer was thermal vacuum deposited thereon as follows. As the light emitting layer, TCz1, a host, was doped with Flrpic, a blue phosphorescent dopant, by 8%, and deposited to 300 Å. After forming an electron transfer layer to 400 Å using TmPyPB, a compound described as the following Bphen was doped with Cs₂CO₃ by 20 wt % to fo m an N-type charge generation layer to 100 Å. As for the second stack, MoO₃ was thermal vacuum deposited first to a thickness of 50 Å to form a P-type charge generation layer. After that, a hole transfer layer was formed to 100 Å on the P-type charge generation layer by doping MoO₃ to TAPC by 20 wt %, and then depositing TAPC to 300 Å.

A light emitting layer was formed thereon by doping Ir(ppy)₃, a green phosphorescent dopant, to TCz1, a host, by 8 wt %, and depositing the result to 300 Å, and then an electron transfer layer was formed to 600 Å using TmPyPB.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured (Comparative Example 1).

Meanwhile, all the organic compounds required to manufacture the OLED were vacuum sublimation purified under 10⁻⁸ torr to 10⁻⁶ torr for each material to be used in the OLED manufacture.

Organic light emitting devices (Examples 1 to 10 and Comparative Examples 2 to 4) were manufactured in the same manner as in Experimental Example 1 except that compounds shown in the following Table 4 were used instead of MoO₃ used when forming the second stack P-type charge generation layer.

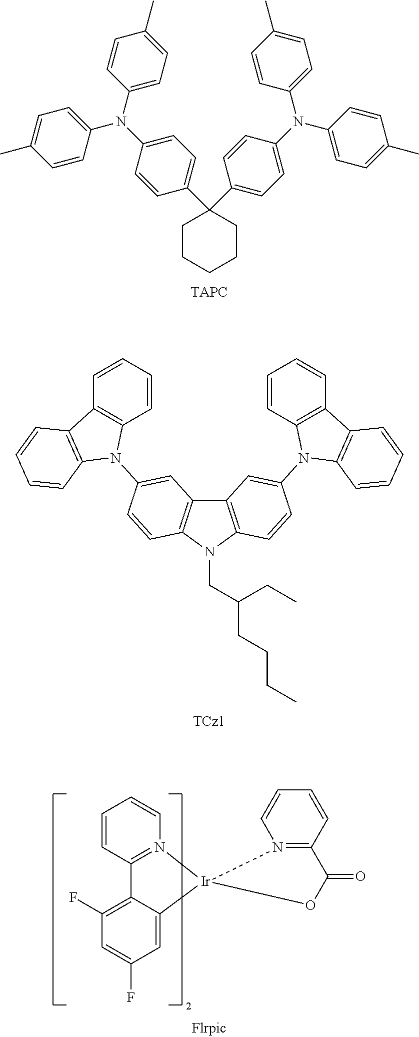

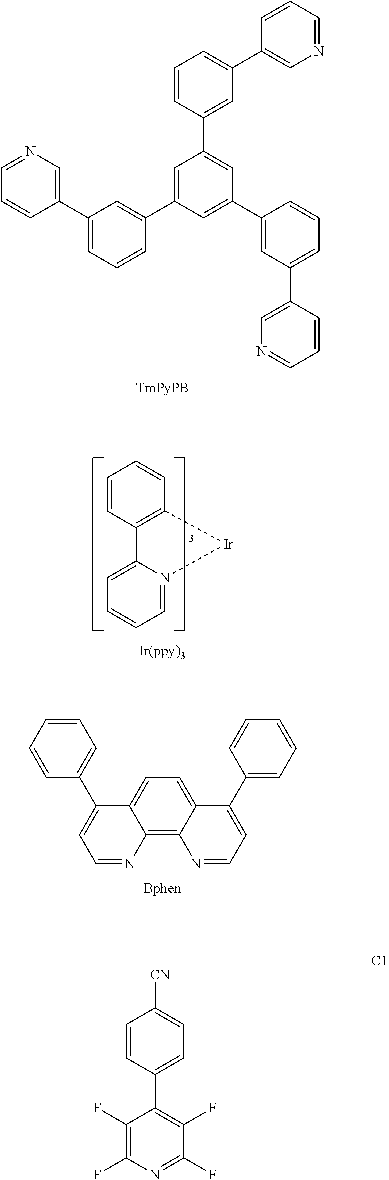

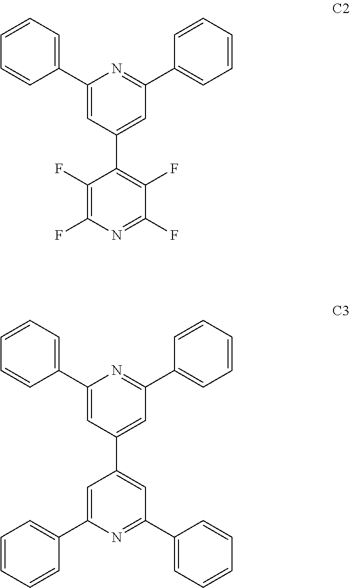 [SMILES: Fc1nc(F)c(F)c(-c2cc(-c3ccccc3)nc(-c3ccccc3)c2)c1F ; c1ccc(-c2cc(-c3cc(-c4ccccc4)nc(-c4ccccc4)c3)cc(-c3ccccc3)n2)cc1]

2) Driving Voltage and Light Emission Efficiency of Organic Light Emitting Device

For each of the organic light emitting devices of Examples 1 to 6 and Comparative Example 1 manufactured as above, electroluminescent (EL) properties were measured using M7000 manufactured by McScience Inc., and with the measurement results, 195 was measured when standard luminance was 3,500 cd/m2 through a lifetime measurement system (M6000) manufactured by McScience Inc. 195 means a lifetime (unit: h, time), a time taken to become 95% with respect to initial luminance.

Results of measuring driving voltage, light emission efficiency, external quantum efficiency and color coordinate (CIE) of the white organic light emitting devices manufactured according to the present disclosure are as shown in the following Table 4.

TABLE 4

| | | Driving | | | Life- |
|---|---|---|---|---|---|
| | Com- | Voltage | Efficiency | | time |
| | pound | (V) | (cd/A) | CIE (x, y) | (T95) |
| Example 1 | 1 | 7.13 | 68.18 | (0.219, 0.434) | 87 |
| Example 2 | 2 | 7.01 | 69.86 | (0.220, 0.420) | 83 |
| Example 3 | 4 | 7.07. | 67.07 | (0.221, 0.423) | 80 |
| Example 4 | 6 | 7.08 | 68.11 | (0.224, 0.433) | 84 |
| Example 5 | 9 | 7.19 | 66.29 | (0.211, 0.424) | 85 |
| Example 6 | 11 | 7.02 | 68.77 | (0.220, 0.420) | 81 |
| Example 7 | 20 | 7.06 | 67.41 | (0.221, 0.426) | 77 |
| Example 8 | 17 | 7.11 | 67.16 | (0.208, 0.428) | 79 |
| Example 9 | 33 | 7.20 | 65.44 | (0.217, 0.438) | 76 |
| Example 10 | 38 | 7.23 | 65.37 | (0.206, 0.439) | 79 |
| Comparative | MoO₃ | 8.15 | 55.06 | (0.212, 0.430) | 62 |
| Example 1 | | | | | |
| Comparative | C1 | 8.07 | 57.33 | (0.214, 0.422) | 64 |
| Example 2 | | | | | |
| Comparative | C2 | 8.00 | 58.10 | (0.215, 0.423) | 62 |
| Example 3 | | | | | |
| Comparative | C3 | 8.11 | 58.50 | (0.220, 0,429) | 66 |
| Example 4 | | | | | |

As seen from the results of Table 4, the organic light emitting device using the P-type charge generation layer material of the 2-stack white organic light emitting device of the present disclosure had lower driving voltage and improved light emission efficiency compared to Comparative Examples 1 to 4.

Such a result is considered to be due to the fact that, by using the compound of the present disclosure in the P-type charge generation layer, holes are smoothly injected by the charge generation layer being formed with materials having a similar energy level as the energy level of the hole transfer layer, and electrons produced from the P-type charge generation layer anionized and stabilized are readily injected through a gap state produced in the N-type charge generation layer. In addition, it is considered that the P-type charge generation layer readily injects and transfers electrons to the N-type charge generation layer by having a low LUMO energy level, and as a result, the organic light emitting device had lowered driving voltage and improved efficiency and lifetime.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

1. A heterocyclic compound represented by the following Chemical Formula 1: 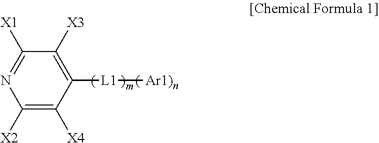
wherein, in Chemical Formula 1,
L1 is a direct bond; a substituted or unsubstituted arylene group having 6 to 60 carbon atoms; or a substituted or unsubstituted heteroarylene group having 2 to 60 carbon atoms;
two of X1 to X4 are each independently an aryl group having 6 to 40 carbon atoms unsubstituted or substituted with one or more selected from the group consisting of a halogen group, a cyano group, a haloalkyl group and −NO₂; or a heteroaryl group having 2 to 40 carbon atoms unsubstituted or substituted with one or more selected from the group consisting of a halogen group, a cyano group, a haloalkyl group and −NO₂, and the remaining two are each independently a halogen group; a cyano group; or −NO₂;
Ar1 is an aryl group having 6 to 60 carbon atoms unsubstituted or substituted with one or more selected from the group consisting of a halogen group, a cyano group, a haloalkyl group and −NO₂; or a heteroaryl group having 2 to 60 carbon atoms unsubstituted or substituted with one or more selected from the group consisting of a halogen group, a cyano group, a haloalkyl group and −NO₂; and
m is an integer of 0 to 2, n is an integer of 1 or 2, and when m and n are each 2, substituents in the parentheses are the same as or different from each other.

2. The heterocyclic compound of claim 1, wherein Chemical Formula 1 is represented by the following Chemical Formula 2 or 3: 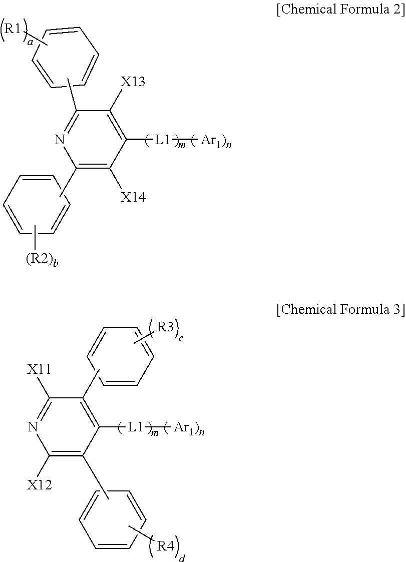
in Chemical Formulae 2 and 3,
L1, Ar1, m and n have the same definitions as in Chemical Formula 1;
X11 to X14 and R1 to R4 are the same as or different from each other, and each independently a halogen group; a cyano group; a haloalkyl group; or −NO₂; and
a to d are each independently an integer of 1 to 5, and when a to d are each 2 or greater, substituents in the parentheses are the same as or different from each other.

3. The heterocyclic compound of claim 1, wherein Chemical Formula 1 is represented by the following Chemical Formula 4 or 5: 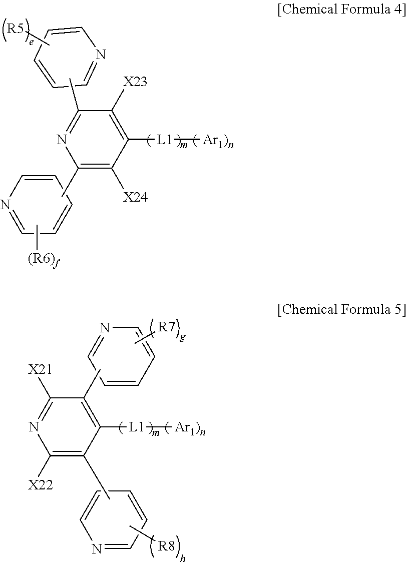
in Chemical Formulae 4 and 5,
L1, Ar1, m and n have the same definitions as in Chemical Formula 1;
X21 to X24 are the same as or different from each other, and each independently a halogen group; a cyano group; a haloalkyl group; or −NO₂;
R5 to R8 are the same as or different from each other, and each independently hydrogen;
deuterium; a halogen group; a cyano group; a haloalkyl group; or −NO₂; and
e to h are each independently an integer of 1 to 4, and when e to h are each 2 or greater, substituents in the parentheses are the same as or different from each other.

4. The heterocyclic compound of claim 1, wherein Chemical Formula 1 is represented by any one of the following compounds:

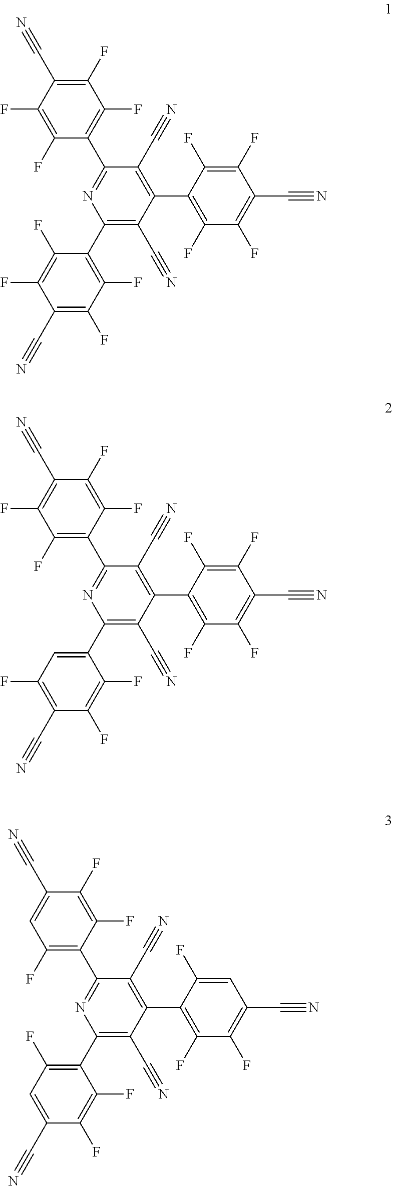 [SMILES: N#Cc1c(F)c(F)c(-c2nc(-c3c(F)c(F)c(C#N)c(F)c3F)c(C#N)c(-c3c(F)c(F)c(C#N)c(F)c3F)c2C#N)c(F)c1F ; N#Cc1c(F)cc(-c2nc(-c3c(F)c(F)c(C#N)c(F)c3F)c(C#N)c(-c3c(F)c(F)c(C#N)c(F)c3F)c2C#N)c(F)c1F ; N#Cc1cc(F)c(-c2nc(-c3c(F)cc(C#N)c(F)c3F)c(C#N)c(-c3c(F)cc(C#N)c(F)c3F)c2C#N)c(F)c1F]

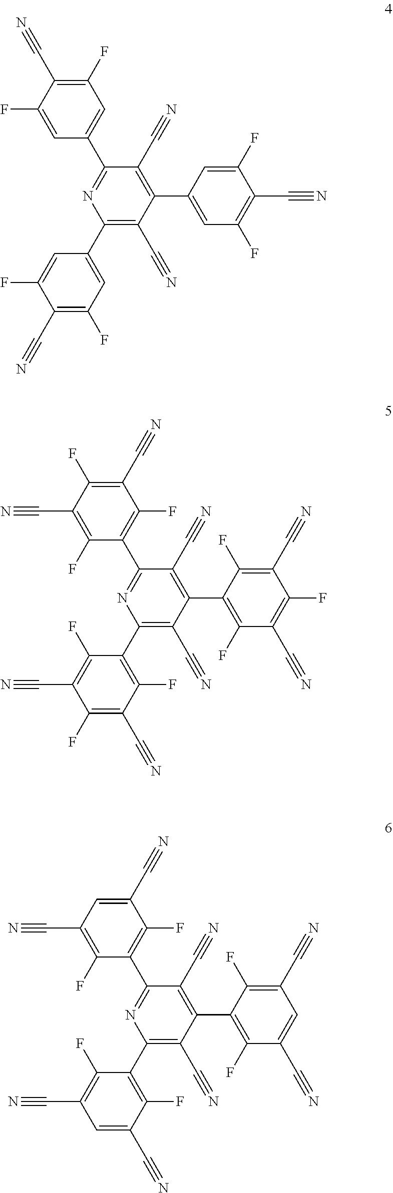 [SMILES: N#Cc1c(F)cc(-c2nc(-c3cc(F)c(C#N)c(F)c3)c(C#N)c(-c3cc(F)c(C#N)c(F)c3)c2C#N)cc1F ; N#Cc1c(F)c(C#N)c(F)c(-c2nc(-c3c(F)c(C#N)c(F)c(C#N)c3F)c(C#N)c(-c3c(F)c(C#N)c(F)c(C#N)c3F)c2C#N)c1F ; N#Cc1cc(C#N)c(F)c(-c2nc(-c3c(F)c(C#N)cc(C#N)c3F)c(C#N)c(-c3c(F)c(C#N)cc(C#N)c3F)c2C#N)c1F]

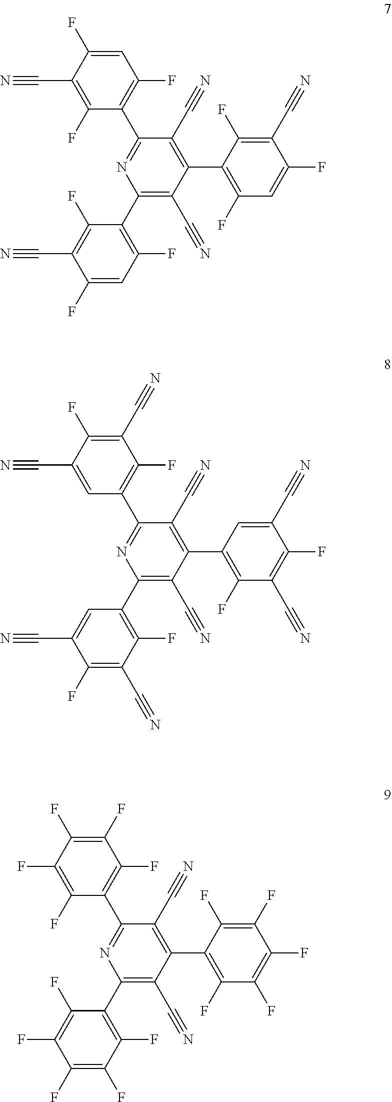 [SMILES: N#Cc1c(F)cc(F)c(-c2nc(-c3c(F)cc(F)c(C#N)c3F)c(C#N)c(-c3c(F)cc(F)c(C#N)c3F)c2C#N)c1F ; N#Cc1cc(-c2nc(-c3cc(C#N)c(F)c(C#N)c3F)c(C#N)c(-c3cc(C#N)c(F)c(C#N)c3F)c2C#N)c(F)c(C#N)c1F ; N#Cc1c(-c2c(F)c(F)c(F)c(F)c2F)nc(-c2c(F)c(F)c(F)c(F)c2F)c(C#N)c1-c1c(F)c(F)c(F)c(F)c1F]

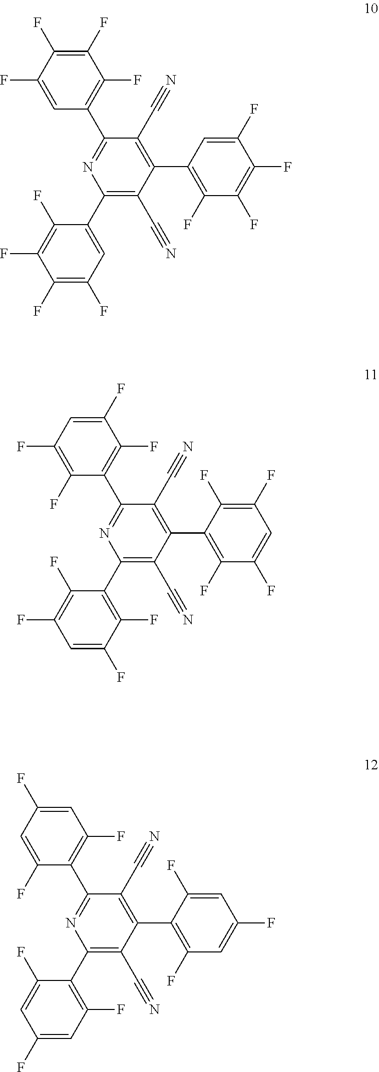 [SMILES: N#Cc1c(-c2cc(F)c(F)c(F)c2F)nc(-c2cc(F)c(F)c(F)c2F)c(C#N)c1-c1cc(F)c(F)c(F)c1F ; N#Cc1c(-c2c(F)c(F)cc(F)c2F)nc(-c2c(F)c(F)cc(F)c2F)c(C#N)c1-c1c(F)c(F)cc(F)c1F ; N#Cc1c(-c2c(F)cc(F)cc2F)nc(-c2c(F)cc(F)cc2F)c(C#N)c1-c1c(F)cc(F)cc1F]

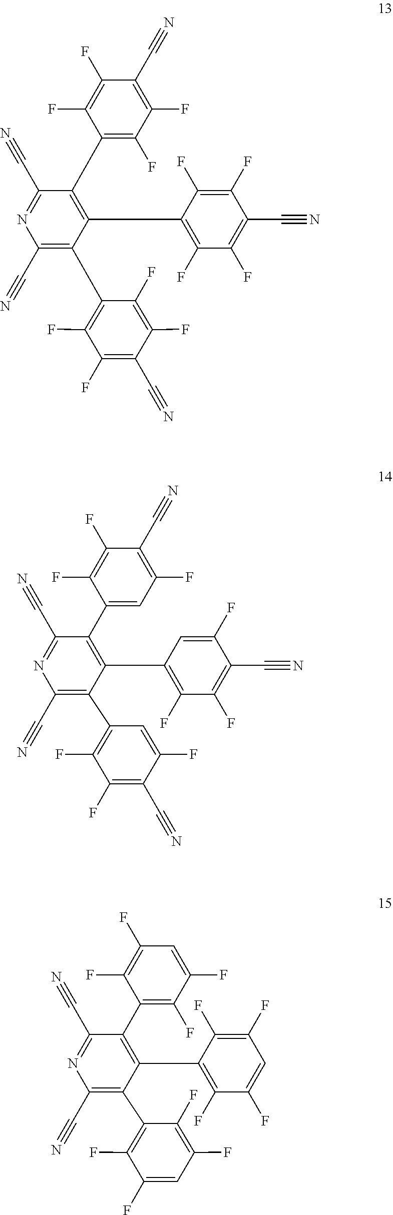 [SMILES: N#Cc1nc(C#N)c(-c2c(F)c(F)c(C#N)c(F)c2F)c(-c2c(F)c(F)c(C#N)c(F)c2F)c1-c1c(F)c(F)c(C#N)c(F)c1F ; N#Cc1nc(C#N)c(-c2cc(F)c(C#N)c(F)c2F)c(-c2cc(F)c(C#N)c(F)c2F)c1-c1cc(F)c(C#N)c(F)c1F ; N#Cc1nc(C#N)c(-c2c(F)c(F)cc(F)c2F)c(-c2c(F)c(F)cc(F)c2F)c1-c1c(F)c(F)cc(F)c1F]

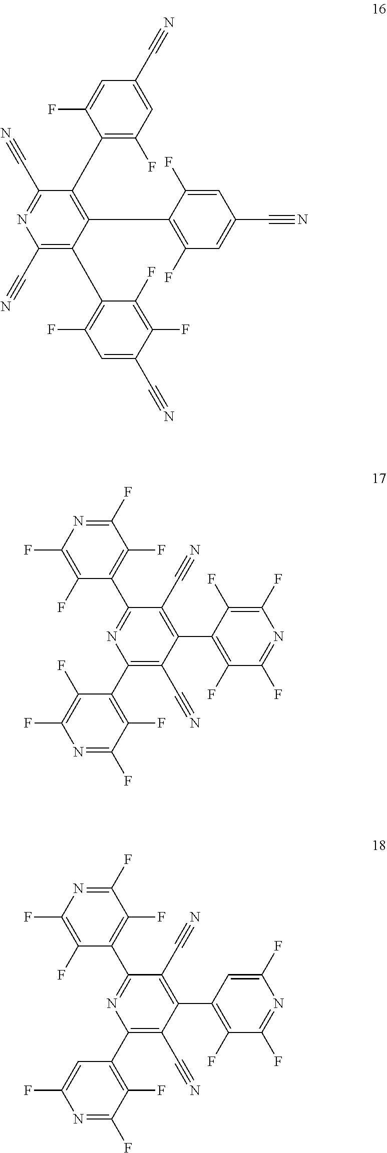 [SMILES: N#Cc1cc(F)c(-c2c(C#N)nc(C#N)c(-c3c(F)cc(C#N)c(F)c3F)c2-c2c(F)cc(C#N)cc2F)c(F)c1 ; N#Cc1c(-c2c(F)c(F)nc(F)c2F)nc(-c2c(F)c(F)nc(F)c2F)c(C#N)c1-c1c(F)c(F)nc(F)c1F ; N#Cc1c(-c2cc(F)nc(F)c2F)nc(-c2c(F)c(F)nc(F)c2F)c(C#N)c1-c1cc(F)nc(F)c1F]

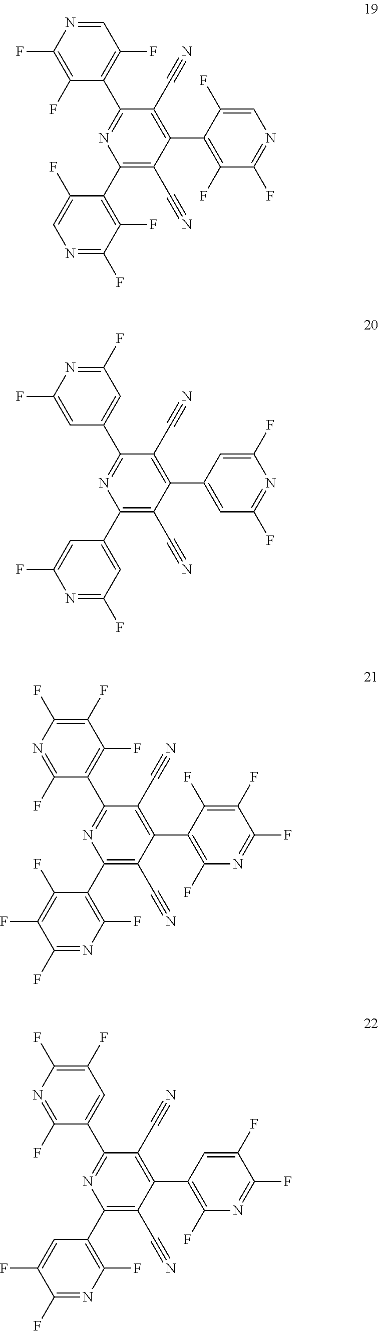 [SMILES: N#Cc1c(-c2c(F)cnc(F)c2F)nc(-c2c(F)cnc(F)c2F)c(C#N)c1-c1c(F)cnc(F)c1F ; N#Cc1c(-c2cc(F)nc(F)c2)nc(-c2cc(F)nc(F)c2)c(C#N)c1-c1cc(F)nc(F)c1 ; N#Cc1c(-c2c(F)nc(F)c(F)c2F)nc(-c2c(F)nc(F)c(F)c2F)c(C#N)c1-c1c(F)nc(F)c(F)c1F ; N#Cc1c(-c2cc(F)c(F)nc2F)nc(-c2cc(F)c(F)nc2F)c(C#N)c1-c1cc(F)c(F)nc1F]

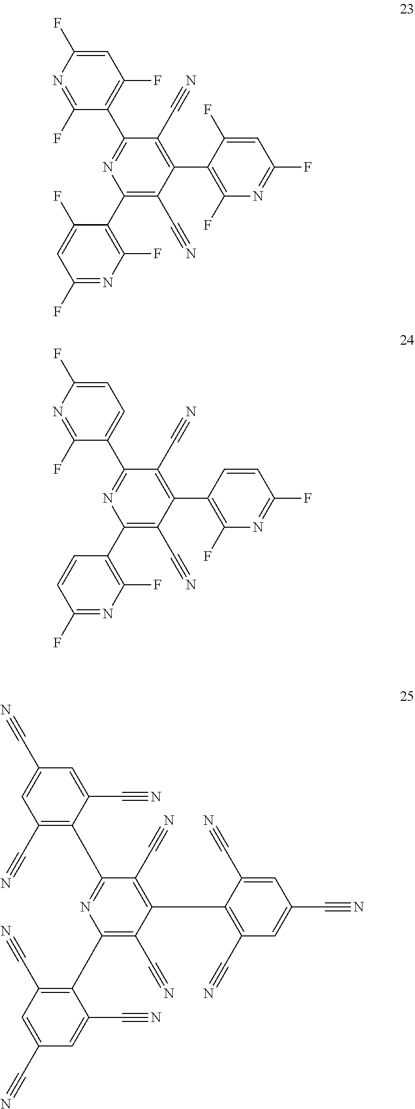

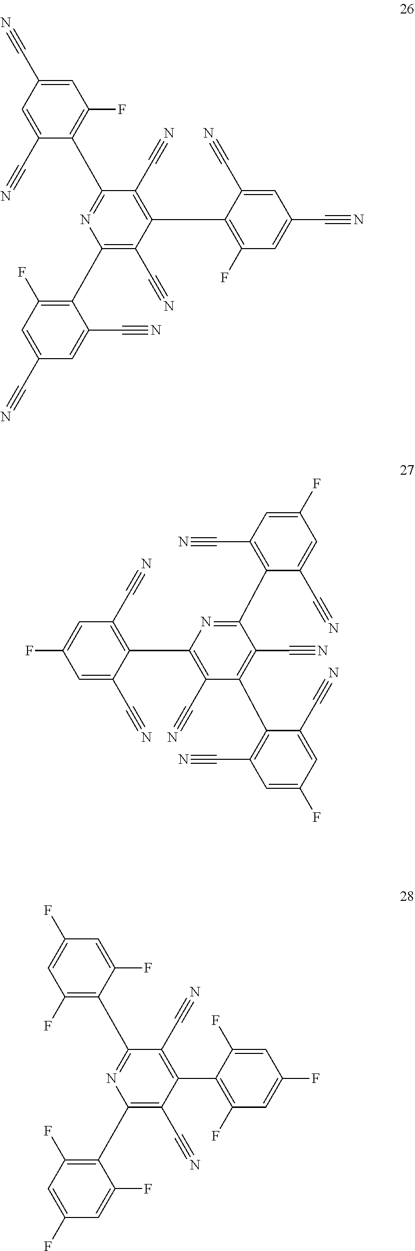 [SMILES: N#Cc1cc(F)c(-c2nc(-c3c(F)cc(C#N)cc3C#N)c(C#N)c(-c3c(F)cc(C#N)cc3C#N)c2C#N)c(C#N)c1 ; N#Cc1cc(F)cc(C#N)c1-c1nc(-c2c(C#N)cc(F)cc2C#N)c(C#N)c(-c2c(C#N)cc(F)cc2C#N)c1C#N ; N#Cc1c(-c2c(F)cc(F)cc2F)nc(-c2c(F)cc(F)cc2F)c(C#N)c1-c1c(F)cc(F)cc1F]

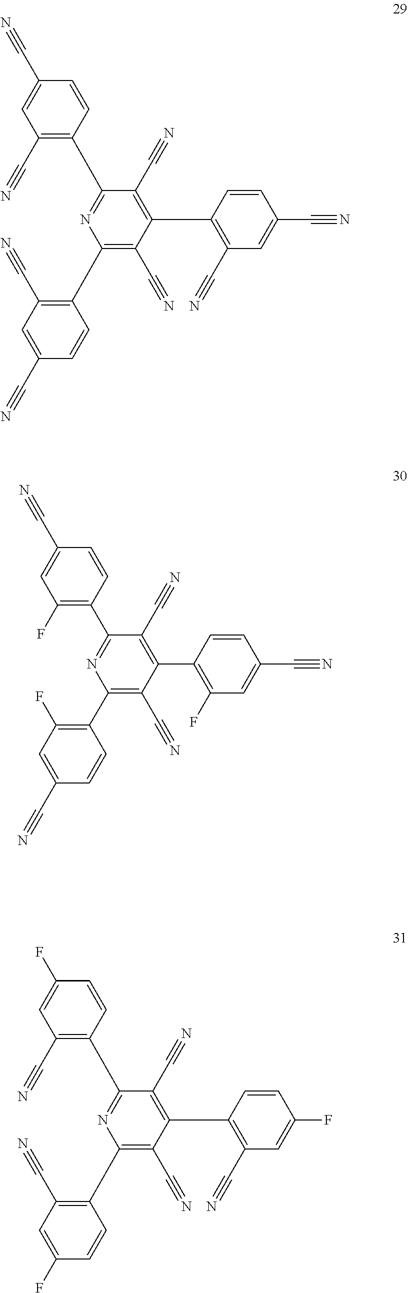 [SMILES: N#Cc1ccc(-c2nc(-c3ccc(C#N)cc3C#N)c(C#N)c(-c3ccc(C#N)cc3C#N)c2C#N)c(C#N)c1 ; N#Cc1ccc(-c2nc(-c3ccc(C#N)cc3F)c(C#N)c(-c3ccc(C#N)cc3F)c2C#N)c(F)c1 ; N#Cc1cc(F)ccc1-c1nc(-c2ccc(F)cc2C#N)c(C#N)c(-c2ccc(F)cc2C#N)c1C#N]

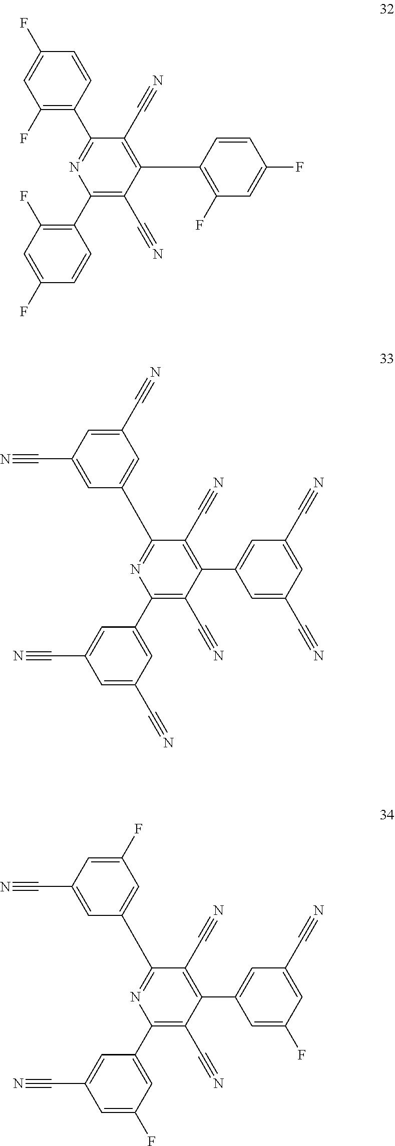 [SMILES: N#Cc1c(-c2ccc(F)cc2F)nc(-c2ccc(F)cc2F)c(C#N)c1-c1ccc(F)cc1F ; N#Cc1cc(C#N)cc(-c2nc(-c3cc(C#N)cc(C#N)c3)c(C#N)c(-c3cc(C#N)cc(C#N)c3)c2C#N)c1 ; N#Cc1cc(F)cc(-c2nc(-c3cc(F)cc(C#N)c3)c(C#N)c(-c3cc(F)cc(C#N)c3)c2C#N)c1]

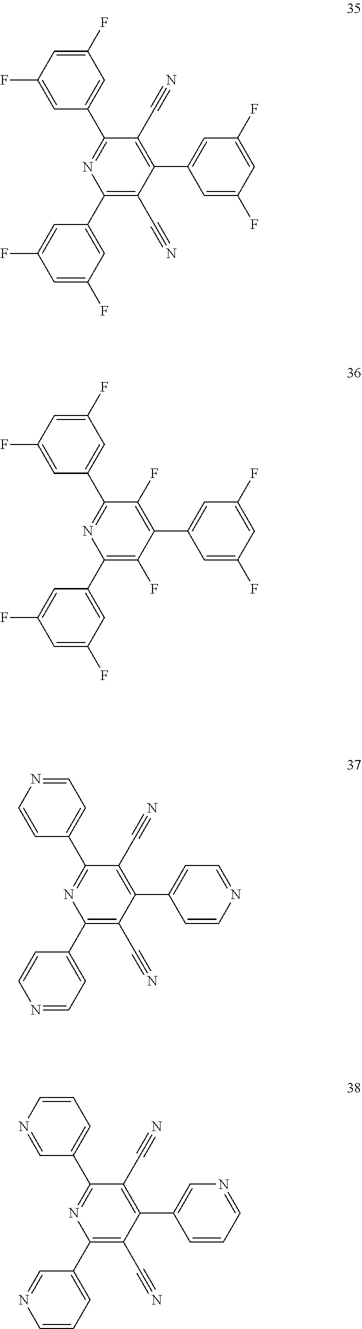 [SMILES: N#Cc1c(-c2cc(F)cc(F)c2)nc(-c2cc(F)cc(F)c2)c(C#N)c1-c1cc(F)cc(F)c1 ; Fc1cc(F)cc(-c2nc(-c3cc(F)cc(F)c3)c(F)c(-c3cc(F)cc(F)c3)c2F)c1 ; N#Cc1c(-c2ccncc2)nc(-c2ccncc2)c(C#N)c1-c1ccncc1 ; N#Cc1c(-c2cccnc2)nc(-c2cccnc2)c(C#N)c1-c1cccnc1]

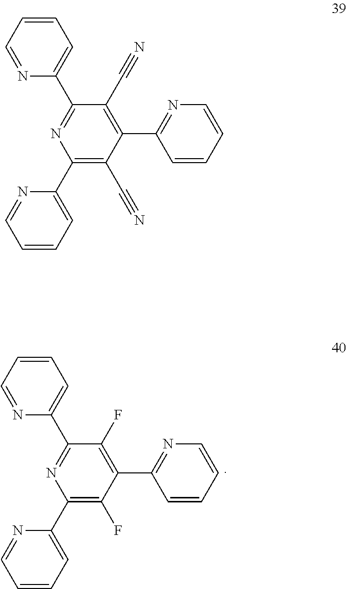 [SMILES: N#Cc1c(-c2ccccn2)nc(-c2ccccn2)c(C#N)c1-c1ccccn1 ; Fc1c(-c2ccccn2)nc(-c2ccccn2)c(F)c1-c1ccccn1]

5. An organic light emitting device comprising:
a first electrode;
a second electrode; and
one or more organic material layers provided between the first electrode and the second electrode,
wherein one or more layers of the organic material layers comprise the heterocyclic compound of claim 1.

6. The organic light emitting device of claim 5, wherein the organic material layer comprises a hole injection layer, and the hole injection layer comprises the heterocyclic compound.

7. The organic light emitting device of claim 5, comprising:
a first stack provided on the first electrode and comprising a first light emitting layer;
a charge generation layer provided on the first stack;
a second stack provided on the charge generation layer and comprising a second light emitting layer; and
the second electrode provided on the second stack.

8. The organic light emitting device of claim 7, wherein the charge generation layer comprises the heterocyclic compound.
